(19) United States
(12) Patent Application Publication
(10) Pub. No.: US 2004/0138104 A1
(43) Pub. Date: Jul. 15, 2004

(54) Peptides

(75) Inventors: Terrence R. Burke JR., Bethesda, MD (US); Kyeong Lee, Frederick, MD (US); Yang Gao, Branford, CT (US); Jason Phan, Walkersville, MD (US); David S. Waugh, Walkersville, MD (US)

Correspondence Address: LEYDIG VOIT & MAYER, LTD, 700 THIRTEENTH ST. NW, SUITE 300, WASHINGTON, DC, 20005-3960, US

(73) Assignees: The Government of the United States of America represented by the Secretary,; Department of Health and Human Services, Rockville, MD

(21) Appl. No.: 10/341,607

(22) Filed: Jan. 14, 2003

(51) Int. Cl. A61K 38/05; C07K 5/06

(52) U.S. Cl. 514/7; 530/331

(57) ABSTRACT

Disclosed are compounds which are tripeptides of the formula P-A-B-C or prodrugs thereof, wherein A is an amino acid having a carboxy alkyl group (e.g., carboxy C₁-C₆ alkyl group), B is tyrosine, phenylalanine, or a substituted tyrosine or phenylalanine, C is a hydrophobic amino acid, and P is an amine protecting group protecting the amine end of A. Also disclosed are pharmaceutical compositions comprising such a compound and a pharmaceutically acceptable carrier, and a method of treating an animal, e.g., a human, exposed to or infected by Yersinia pestis. The compounds find use as anti-bioterrorism agents.

### FIELD OF THE INVENTION

This invention pertains to compounds, i.e., peptides or prodrugs thereof, which are useful, in general, as inhibitors of the phosphotyrosine phosphatase enzyme, and in particular, as inhibitors of the Yersinia phosphatase (YopH) enzyme. The invention also provides pharmaceutical compositions and a method of inhibiting the YopH enzyme as well as a method of treating plague or Black Death. The compounds may be useful as anti-bioterrorism agents.

### BACKGROUND OF THE INVENTION

Protein tyrosine phosphatases (PTPases) catalyze the hydrolysis of phosphotyrosine residues during signal transduction. These enzymes, along with the protein tyrosine kinases, play a central role in regulating cell growth, differentiation, and metabolism. An example of such a PTPase is the YopH protein in Yersinia pestis which is responsible for causing plague. Plague is a “zoonotic” disease which spreads to all organs of the body via a path leading from the lymphatic system to the lymph nodes and finally to the blood. Common syndromes include bubonic, pneumonic and septicemic plague. Yersinia pestis is considered as a possible agent for use in bioterrorism. Sifton, D. W. et al., Section 1: Biological Agents. Plague, pp. 22-23 in PDR Guide to Biological and Chemical Warfare Response; Thomson/Physician's Desk Reference, Inc., Montvale, N.J. (2002); Cornelis, G. R., Molecular and Biological Aspects of Plague; Proc. Natl. Acad. Sci. USA, 97, 8778-83 (2000).

Within Yersinia is a 70 kb virulence plasmid which encodes for a system of secreted proteins called “Yops” that fall into two categories: intracellular effectors and translocators. Among the effectors is the YopH protein, which is a phosphotyrosine phosphatase that plays an antiphagocytic role by dephosphorylating focal adhesion proteins. Black, D. S., et al., The Yersinia Tyrosine Phosphatase YopH targets a novel adhesion-regulated signaling complex in macrophages. Cell. Microbiol. 2, 401-414 (2000). It has been demonstrated that the phosphatase activity is required for bacterial pathogenesis. Blistra, J. J. et al., Tyrosine Phosphatase hydrolysis of host proteins by an essential Yersinia virulence determinant. J. Biol. Chem., 88, 1187-91 (1991).

Another example of a PTPase is PTP1B which mediates type II diabetes. Chen et al., Bioorg. & Med. Chem. Lett., 11, 1935-1938 (2001). This phosphatase is a negative regulator of insulin-dependent signaling, and likely acts by dephosporylating the insulin receptor.

The foregoing shows that there exists a need for PTPase inhibitors, particularly inhibitors of YopH and PTP1B. There exists a need for treating Yersinia infections. There exists a need for anti-bioterrorism agents. There further exists a need for treating diabetes. The advantages of the invention, as well as additional inventive features, will be apparent from the description of the invention provided herein.

### BRIEF SUMMARY OF THE INVENTION

The foregoing needs have been fulfilled to a great extent by the present invention which provides a compound which is a tripeptide of the formula P-A-B-C or a prodrug thereof, wherein A is an amino acid having a carboxy alkyl group (e.g., carboxy C₁-C₆ alkyl group), B is tyrosine, phenylalanine, or a substituted tyrosine or phenylalanine, C is a hydrophobic amino acid, and P is an amine protecting group protecting the amine end of A. The present invention further provides pharmaceutical compositions comprising a compound described above and a pharmaceutically acceptable carrier. The present invention also provides a method of treating an animal, e.g., a human, exposed to or afflicted by Yersinia pestis. The present invention also provides a method for inhibiting a PTP enzyme. The present invention further provides a method of treating diseases such as those associated with immune dysfunction, cancer, and diabetes. The compounds of the present invention reduce or eliminate the toxicity of the YopH enzyme.

While the invention has been described and disclosed below in connection with certain embodiments and procedures, it is not intended to limit the invention to those specific embodiments. Rather it is intended to cover all such alternative embodiments and modifications as fall within the spirit and scope of the invention.

### BRIEF DESCRIPTION OF THE DRAWINGS

FIG. 1A depicts a reaction scheme employed in the synthesis of some compounds in accordance with an embodiment of the present invention. The reagents used are as follows: a) 20% piperidine/NMP; b) i) Fmoc-Leu-OH, DIPCDI, HOBt, NMP; (ii) 20% piperidine/NMP; c) i) Fmoc-pTyr mimetic-OH, DIPCDI, HOBt, NMP, or Fmoc-pTyr mimetic-OH, BOP, HOBt, NMP for TB 37, ii) 20% piperidine/NMP; d) i) (HFPyr, NMP:THF=1:1 for TB32-33), Fmoc-Glu(Ot-Bu)-OH or Fmoc-Glu(OBn)OH, DIPCDI, HOBt, NMP; e) TFA-TES-H₂O.

FIG. 1B depicts a reaction scheme employed in the synthesis of some other compounds in accordance with an embodiment of the present invention. The reagents used are as follows: a) (i) GBZ-Glu (OBn)-OH, ii) TFA-TES-H₂O; b) i) Fmoc-Glu (OBn)-OH, DIPCDI, HOBt, NMP; ii) 20% piperidine/NMP, c) i) 1B.3, NMM, NMP; ii) TFA-TES-H₂O.

FIG. 2 depicts the structures of pTyr mimetics employed in the synthesis of some compounds in accordance with an embodiment of the present invention.

FIG. 3 depicts the structures of some compounds in accordance with an embodiment of the present invention.

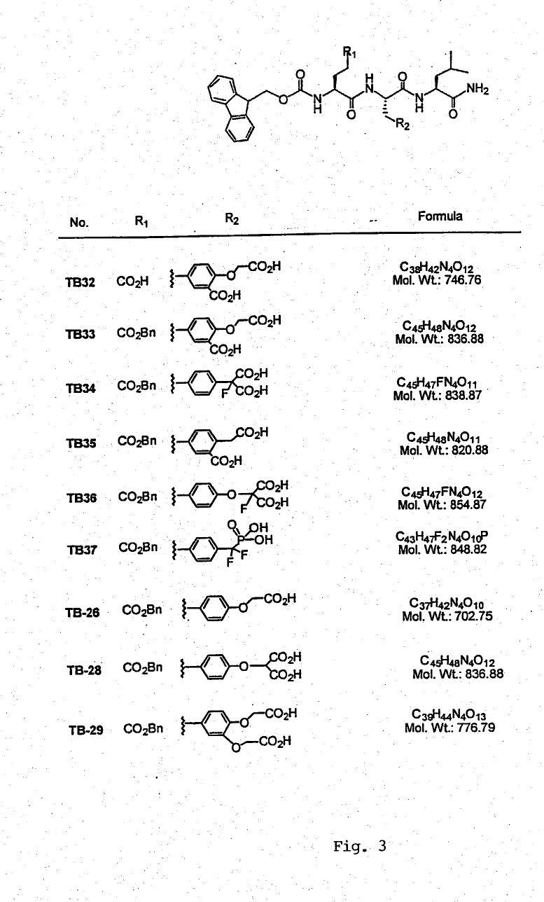

FIG. 4 depicts the structures of some other compounds in accordance with an embodiment of the present invention.

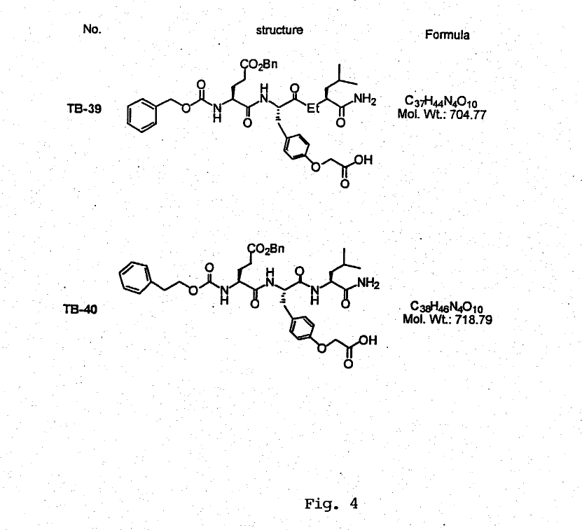

### DETAILED DESCRIPTION OF THE INVENTION

The present invention provides compounds which inhibit phosphotyrosine phosphatases and find use in treating and/or preventing a number of diseases. In particular, the present invention provides compounds which inhibit the Yersinia phosphatase YopH and/or the human phosphatase PTP1B.

The present invention provides a compound which is a tripeptide of the formula P-A-B-C or a prodrug thereof, wherein A is an amino acid having a carboxy C₁-C₆ alkyl group, B is tyrosine, phenylalanine, or a substituted tyrosine or phenylalanine, C is a hydrophobic amino acid, and P is an amine protecting group protecting the amine end of A. In a preferred embodiment, the present invention provides a compound which is a tripeptide of the formula P-A-B-C or a prodrug thereof, wherein A is an amino acid having a carboxy C₁-C₆ alkyl group, B is tyrosine, phenylalanine, or a substituted tyrosine or phenylalanine, C is a hydrophobic amino acid, and P is an amine protecting group protecting the amine end of A; with the proviso that when A is Glu, P is fluorenylmethoxy carbonyl (Fmoc), B is p-phosphonomethyl, p-fluorophosphonomethyl, or p-difluorophosphonomethyl phenylalanine, or p-O-malonyl tyrosine, and C is Ala with a carboxylic acid or amide terminus, the carboxyalkyl group of A is not in acid form.

The carboxyl group of the amino acid A, e.g., carboxy C₁-C₆ alkyl group, can be in any suitable form, for example, in the form of an ester, amide, carbonate, or urethane, preferably an ester. The ester can be a C₁-C₆ alkyl ester, aryl ester, aryl C₁-C₆ alkyl ester, C₁-C₆ alkyl aryl ester, hydroxy C₁-C₆ alkyl ester, halo C₁-C₆ alkyl ester, C₁-C₆ alkoxy C₁-C₆ alkyl ester, C₅-C₈ cycloalkyl ester, C₅-C₈ cyclic amine ester, C₁-C₆ alkanoyloxy C₁-C₆ alkyl ester, C₁-C₆ alkoxy carbonyloxy C₁-C₆ alkyl ester, cycloalkyl carbonyloxy C₁-C₆ alkyl ester, or 1,3-dioxolen-2-onyl C₁-C₆ alkyl ester, wherein the alkyl, cycloalkyl, and cyclic amino group may be optionally substituted by one or more of phenyl, heterocyclyl, C₁-C₆ alkyl, amino, C₁-C₆ alkylamino, C₁-C₆ dialkylamino, hydroxy, C₁-C₆ alkoxy, aryloxy, and benzyloxy. Particular examples of the ester are aryl C₁-C₆ alkyl ester, preferably a benzyl ester.

The amino acid A preferably has carboxy C₁-C₃ alkyl group. Particular examples of A include Glu and Asp.

In an embodiment, B is tyrosine or a substituted tyrosine. The substituted tyrosine, for example, is one where the tyrosyl hydroxyl group and/or a ring hydrogen has been substituted with one, two, or more substituents selected from the group consisting of phosphono C₁-C₆ alkyl, phospho, phospho C₁-C₆ alkyl, phosphono halo C₁-C₆ alkyl, phosphono dihalo C₁-C₆ alkyl, carboxy C₁-C₆ alkyl, carboxy halo C₁-C₆ alkyl, carboxy C₁-C₆ alkoxy, carboxy, dicarboxy C₁-C₆ alkyl, dicarboxy halo C₁-C₆ alkyl, dicarboxy C₁-C₆ alkoxy, dicarboxy halo C₁-C₆ alkoxy, amino, amido, oxalylamino, C₁-C₆ alkylcarbonylamino, sulfo, sulfonyl, C₁-C₆ alkylthio, C₁-C₆ alkylsulfonyl, and halo C₁-C₆ alkyl sulfonyl.

In another embodiment, B is phenylalanine or a substituted phenylalanine. The substituted phenylalanine, for example, is a phenylalanine having one, two, or more substituents (e.g., on the phenyl ring) selected from the group consisting of phosphono C₁-C₆ alkyl, phospho, phospho C₁-C₆ alkyl, phosphono halo C₁-C₆ alkyl, phosphono dihalo C₁-C₆ alkyl, carboxy C₁-C₆ alkyl, carboxy halo C₁-C₆ alkyl, carboxy C₁-C₆ alkoxy, carboxy, dicarboxy C₁-C₆ alkyl, dicarboxy halo C₁-C₆ alkyl, dicarboxy C₁-C₆ alkoxy, dicarboxy halo C₁-C₆ alkoxy, amino, amido, oxalylamino, C₁-C₆ alkylcarbonylamino, sulfo, sulfonyl, C₁-C₆ alkylthio, C₁-C₆ alkylsulfonyl, and halo C₁-C₆ alkyl sulfonyl.

In a particular embodiment, A is Glu or Asp and B is a substituted tyrosine. In another particular embodiment, A is Glu or Asp and B is a substituted phenylalanine. For example, the substituted tyrosine is a tyrosine whose hydroxyl group has been replaced or substituted with one, two, or more substituents selected from the group consisting of phosphono C₁-C₆ alkyl, phospho, phospho C₁-C₆ alkyl, phosphono halo C₁-C₆ alkyl, phosphono dihalo C₁-C₆ alkyl, carboxy C₁-C₆ alkyl, carboxy halo C₁-C₆ alkyl, carboxy C₁-C₆ alkoxy, carboxy, dicarboxy C₁-C₆ alkyl, dicarboxy halo C₁-C₆ alkyl, dicarboxy C₁-C₆ alkoxy, dicarboxy halo C₁-C₆ alkoxy, amino, amido, oxalylamino, C₁-C₆ alkylcarbonylamino, sulfo, sulfonyl, C₁-C₆ alkylthio, C₁-C₆ alkylsulfonyl, and halo C₁-C₆ alkyl sulfonyl. When a hydroxyl group is substituted with a substituent, the oxygen atom of the hydroxyl group remains on the phenyl ring.

The substituents on the tyrosine and phenylalanine can be at any suitable position, o-, m-, or p- to the methylene group, preferably the p-position, and in an embodiment, at m- and p-positions. In an embodiment, the tyrosine or phenylalanine has two substituents, for example, a carboxy and a carboxy C₁-C₆ alkoxy, a carboxy and a carboxy C₁-C₆ alkyl, or two carboxy C₁-C₆ alkoxy substituents.

In an embodiment, the present invention provides a compound of the formula P-A-B-C, wherein C is a hydrophobic amino acid having a hydrophobic group selected from the group consisting of C₁-C₆ alkyl, C₁-C₆ haloalkyl, C₅-C₈ cycloalkyl, C₅-C₈ cycloalkyl C₁-C₆ alkyl, C₅-C₈ cycloalkoxy C₁-C₆ alkyl, aryl C₅-C₈ cycloalkyl, aryl, aryl C₁-C₆ alkyl, C₁-C₆ alkyl aryl, heterocyclyl, heterocyclyl C₁-C₆ alkyl, C₁-C₆ alkylthio C₁-C₆ alkyl, C₁-C₆ alkoxy C₁-C₆ alkyl, and hydroxyphenyl C₁-C₆ alkyl. In a particular embodiment, the present invention provides a compound wherein A is Glu or Asp and C is a hydrophobic amino acid having a hydrophobic group selected from the group consisting of C₁-C₆ alkyl, C₁-C₆ haloalkyl, C₅-C₈ cycloalkyl, C₅-C₈ cycloalkyl C₁-C₆ alkyl, C₅-C₈ cycloalkoxy C₁-C₆ alkyl, aryl C₅-C₈ cycloalkyl, aryl, aryl C₁-C₆ alkyl, C₁-C₆ alkyl aryl, heterocyclyl, heterocyclyl C₁-C₆ alkyl, C₁-C₆ alkylthio C₁-C₆ alkyl, C₁-C₆ alkoxy C₁-C₆ alkyl, and hydroxyphenyl C₁-C₆ alkyl. In a preferred embodiment, the present invention provides a compound wherein C is a hydrophobic amino acid having a C₁-C₆ alkyl group. In a preferred embodiment, the carboxyl end of C in the above peptides is carboxamide.

In another preferred embodiment, the present invention provides compounds as described above wherein C is selected from the group consisting of Ala, Val, Ile, Leu, Met, Phe, Tyr, Trp, and Nle, more preferably Ala or Leu. In a further preferred embodiment, the carboxyl end of C in the above compounds is carboxamide.

The amine protecting group (P) can be any suitable protecting group. For example, P is an amine protecting group selected from the group consisting of an aryl C₁-C₆ alkoxycarbonyl, C₁-C₆ alkoxycarbonyl, carbobenzoxy (Cbz), and carbamoyl, preferably an aryl C₁-C₆ alkoxycarbonyl. In a preferred embodiment, the aryl C₁-C₆ alkoxycarbonyl is an aryl C₁-C₃ alkoxycarbonyl, and more preferably a methoxycarbonyl; in another preferred embodiment, the C₁-C₆ alkoxycarbonyl is a C₁-C₄ alkoxycarbonyl, and more preferably t-butoxycarbonyl (tBoc).

Additional examples of amine protecting groups include formyl, aralkyl groups (for example benzyl and substituted benzyl, p-methoxybenzyl, nitrobenzyl and 2,4-dimethoxybenzyl, and triphenylmethyl); di-p-anisylmethyl and furylmethyl groups; lower alkoxycarbonyl (for example t-butoxycarbonyl); lower alkenyloxycarbonyl (for example allyloxycarbonyl); phenyl lower alkoxycarbonyl groups (for example benzyloxycarbonyl, p-methoxybenzyloxycarbonyl, o-nitrobenzyloxycarbonyl, p-nitrobenzyloxycarbonyl; trialkylsilyl (for example trimethylsilyl and t-butyldimethylsilyl); alkylidene (for example methylidene); benzylidene and substituted benzylidene groups. See Protective Groups in Organic Synthesis, 3rd Edition, by Green et al., John Wiley & Sons for general guidance on protecting groups.

The term “aryl” in this application refers to an aromatic group of 1-4 aromatic rings and/or heteroaromatic rings. The aromatic rings can be 5-7-membered rings, preferably 5 and/or 6-membered rings. The term “halo” refers to fluorine, chlorine, bromine, or iodine, and preferably fluorine.

The aryl portion of aryl C₁-C₆ alkoxycarbonyl protecting group can be an aromatic group of 1-4 aromatic rings and/or heteroaromatic rings. The aromatic rings can be 5-7-membered rings, preferably 5 and/or 6-membered rings. The aromatic rings may have one, two, or more substituents, e.g., nitro, alkoxy, alkenyloxy, and allyloxy groups. Examples of suitable aromatic rings include phenyl, naphthyl, anthracenyl, and fluorenyl. Examples of heteroaromatic rings include aromatic rings containing 1, 2, 3, or more hetero atoms, e.g., N, O, and S with or without carbon atoms. Particular examples of P include fluorenylmethoxy (Fmoc), tBoc, and Cbz, preferably Fmoc.

In a specific embodiment, the compound of the present invention has Glu or Asp as A and as P an aryl C₁-C₆ alkoxycarbonyl, preferably Fmoc. In another embodiment, the compound of the present invention has Glu or Asp as A, a substituted tyrosine as B, and Fmoc as P. In yet another embodiment, the compound of the present invention has Glu or Asp as A, a substituted tyrosine as B, Fmoc as P, and Ala or Leu as C. In still another embodiment, the compound of the present invention has Glu or Asp as A, a substituted phenylalanine as B, and Fmoc as P. In yet another embodiment, the compound of the present invention has Glu or Asp as A, a substituted phenylalanine as B, Fmoc as P, and Ala or Leu as C.

In an embodiment, the compound of the present invention has the formula I:

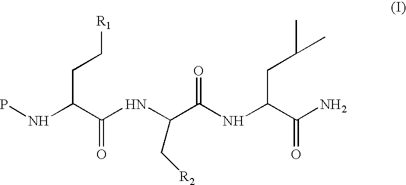

wherein P is selected from the group consisting of aryl C₁-C₆ alkoxy carbonyl, R₁ is aryl C₁-C₆ alkoxy carbonyl or aryloxy carbonyl, and R₂ is selected from the group consisting of carboxyaryl, carboxy C₁-C₆ alkoxy aryl, malonyloxyaryl, dicarboxy C₁-C₆ alkoxy aryl, carboxy-carboxy C₁-C₆ alkoxy aryl, halomalonyl aryl, carboxy-carboxy C₁-C₆ alkyl aryl, halomalonyloxy aryl, and dihalophosphono C₁-C₆ alkyl aryl.

In a preferred embodiment, the present invention provides compounds of formula I, wherein P is selected from the group consisting of Fmoc, benzyloxy carbonyl, and phenylethyloxy carbonyl, R₁ is benzyloxy carbonyl or phenoxy carbonyl, and R₂ is selected from the group consisting of 4-carboxy phenyl, 4-carboxymethoxy phenyl, 4-malonyloxy phenyl, 3,4-dicarboxymethoxy phenyl, 3-carboxy-4-carboxymethoxy phenyl, 4-(α-fluoro malonyl) phenyl, 3-carboxy-4-carboxymethyl phenyl, 4-(α-fluoro malonyloxy) phenyl, and 4-(α,α-difluoro phosphonomethyl) phenyl.

The present invention provides, in an embodiment, the compound of formula Ia:

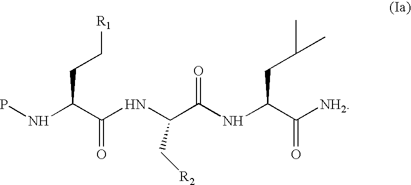

In a preferred embodiment of the compound of formula Ia, P is selected from the group consisting of Fmoc, benzyloxy carbonyl, and phenylethyloxy carbonyl, R₁ is benzyloxy carbonyl or phenoxy carbonyl, and R₂ is selected from the group consisting of 4-carboxy phenyl, 4-carboxymethoxy phenyl, 4-malonyloxy phenyl, 3,4-dicarboxymethoxy phenyl, 3-carboxy-4-carboxymethoxy phenyl, 4-(α-fluoro malonyl) phenyl, 3-carboxy-4-carboxymethyl phenyl, 4-(α-fluoro malonyloxy) phenyl, and 4-(α,α-difluoro phosphonomethyl) phenyl. In a further preferred embodiment, P is Fmoc, R₁ is benzyloxy carbonyl, R₂ is selected from the group consisting of 4-carboxymethoxy phenyl, 3-carboxy-4-carboxymethoxy phenyl, 4-(α-fluoro malonyl) phenyl, 3-carboxy-4-carboxymethyl phenyl, 4-(α-fluoro malonyloxy) phenyl, and 4-(α,α-difluoro phosphonomethyl) phenyl.

Specific examples of the compounds of the present invention include compounds wherein P and R₁ are benzyloxy carbonyl and R₂ is 4-carboxymethoxy phenyl; and P is phenylethoxy carbonyl, R₁ is benzyloxy carbonyl, and R₂ is 4-carboxymethoxy phenyl.

The compounds of the present invention can include the substituents R₁ and R₂ in any suitable configuration, i.e., R, S, or R/S.

When the compound contains a basic moiety it may form pharmaceutically-acceptable salts with a variety of inorganic or organic acids, for example hydrochloric, hydrobromic, sulfuric, phosphoric, trifluoroacetic, citric or maleic acid. A suitable pharmaceutically-acceptable salt of the invention when the compound contains an acidic moiety is an alkali metal salt, for example a sodium or potassium salt, an alkaline earth metal salt, for example a calcium or magnesium salt, an ammonium salt or a salt with an organic base which affords a pharmaceutically-acceptable cation, for example a salt with methylamine, dimethylamine, trimethylamine, piperidine, morpholine or tris-(2-hydroxyethyl)amine.

The present invention further provides compounds comprising prodrugs of the peptides described above. Various forms of prodrugs are well known in the art. For examples of such prodrugs, see: Design of Prodrugs, edited by H. Bundgaard (Elsevier, 1985) and Methods in Enzymology, 42, 309-396, edited by K. Widder et al. (Academic Press, 1985); A Textbook of Drug Design and Development, edited by Krogsgaard-Larsen and H. Bundgaard, Chapter 5 “Design and Application of Prodrugs”, by H. Bundgaard p. 113-191 (1991); H. Bundgaard, Advanced Drug Delivery Reviews, 8, 1-38 (1992); H. Bundgaard, et al., Journal of Pharmaceutical Sciences, 77, 285 (1988); and N. Kakeya, et al., Chem. Pharm. Bull., 32, 692 (1984). The peptides can be converted to prodrugs by those skilled in the art following the teachings of these documents.

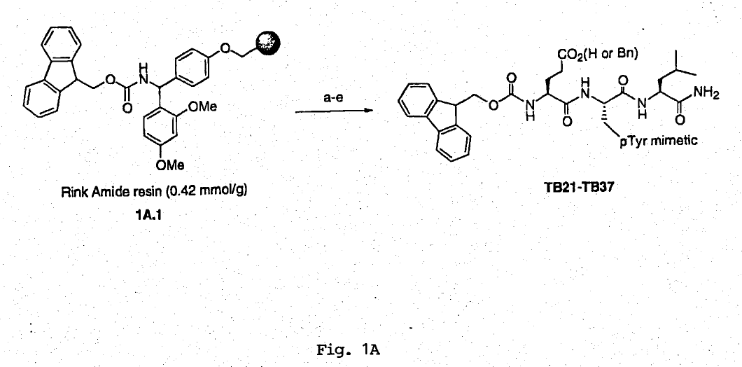

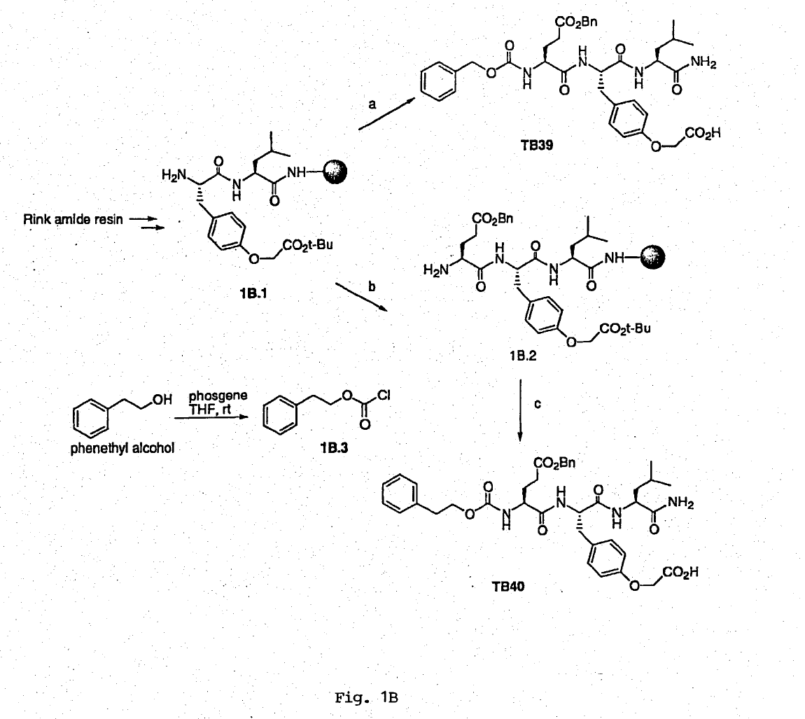

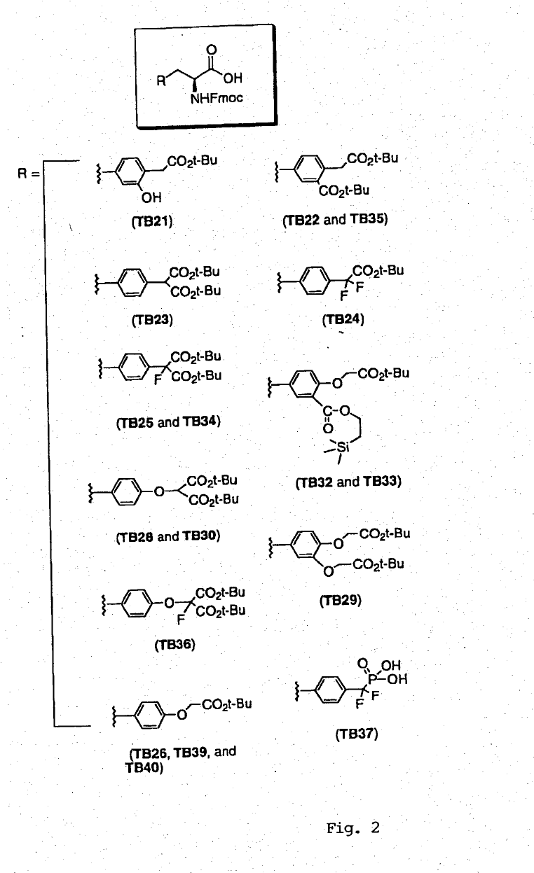

The compounds of the present invention can be prepared by methods generally known in the art. For example, the compounds can be prepared by the solid phase or solution phase peptide synthesis methods. See FIGS. 1-2. The compounds also can be prepared by reacting an amino acid or a peptide with a precursor compound. See, for example, Gao et al., Bioorg. & Med. Chem. Lett., 10, 923-927 (2000); U.S. Pat. Nos. 6,307,090 and 5,200,546; and International Publication WO 00/56760.

The present invention further provides a pharmaceutical composition comprising any one of the compounds described above and a pharmaceutically acceptable carrier. The pharmaceutically acceptable (e.g., pharmacologically acceptable) carriers described herein, for example, vehicles, adjuvants, excipients, or diluents, are well-known to those who are skilled in the art and are readily available to the public. It is preferred that the pharmaceutically acceptable carrier be one which is chemically inert to the active compounds and one which has no detrimental side effects or toxicity under the conditions of use.

The choice of carrier will be determined in part by the particular active agent, as well as by the particular method used to administer the composition. Accordingly, there is a wide variety of suitable formulations of the pharmaceutical composition of the present invention. The following formulations for oral, aerosol, parenteral, subcutaneous, intravenous, intraarterial, intramuscular, interperitoneal, intrathecal, rectal, and vaginal administration are merely exemplary and are in no way limiting.

Formulations suitable for oral administration can comprise (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations can include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

The compounds of the present invention, alone or in combination with other suitable components, can be made into aerosol formulations to be administered via inhalation. These aerosol formulations can be placed into pressurized acceptable propellants, such as dichlorodifluoromethane, propane, nitrogen, and the like. They also can be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations will typically contain from about 0.5 to about 25% by weight of the active ingredient in solution. Suitable preservatives and buffers can be used in such formulations. In order to minimize or eliminate irritation at the site of injection, such compositions may contain one or more nonionic surfactants. The quantity of surfactant in such formulations typically ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The compounds of the present invention may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Trissel, 4th ed., pages 622-630 (1986).

Additionally, the compounds of the present invention may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. For convenience, the total daily dosage may be divided and administered in portions during the day if desired. In proper doses and with suitable administration of certain compounds, the present invention provides for a wide range of responses. Typically the dosages range from about 0.001 to about 1000 mg/kg body weight of the animal being treated/day. Preferred dosages range from about 0.01 to about 10 mg/kg body weight/day, and further preferred dosages range from about 0.01 to about 1 mg/kg body weight/day. The present invention further provides a method of treating an animal, e.g., human, exposed to Yersinia pestis comprising administering to the animal an effective amount of a compound which is a tripeptide of the formula P-A-B-C or a prodrug thereof, wherein A is an amino acid having a carboxy C₁-C₆ alkyl group, B is tyrosine, phenylalanine, or a substituted tyrosine or phenylalanine, C is a hydrophobic amino acid, and P is an amine protecting group protecting the amine end of A.

In a preferred embodiment, the method comprises administering to the animal compound which is a tripeptide of the formula P-A-B-C or a prodrug thereof, wherein A is an amino acid having a carboxy C₁-C₆ alkyl group, B is tyrosine, phenylalanine, or a substituted tyrosine or phenylalanine, C is a hydrophobic amino acid, and P is an amine protecting group protecting the amine end of A; with the proviso that when A is Glu, P is fluorenylmethoxy carbonyl (Fmoc), B is p-phosphonomethyl, p-fluorophosphonomethyl, or p-difluorophosphonomethyl phenylalanine, or p-O-malonyl tyrosine, and C is Ala with a carboxylic acid or amide terminus, the carboxyalkyl group of A is not in acid form.

The present invention further provides a method of inhibiting the protein-tyrosine phosphatase YopH of Yersinia pestis comprising contacting the Yersinia pestis with an effective amount of a compound which is a tripeptide of the formula P-A-B-C or a prodrug thereof, wherein A is an amino acid having a carboxy C₁-C₆ alkyl group, B is tyrosine, phenylalanine, or a substituted tyrosine or phenylalanine, C is a hydrophobic amino acid, and P is an amine protecting group protecting the amine end of A. The contacting is carried out in vivo or in vitro. For example, the compounds can find use as molecular probes as well as in assays to identify, isolate, and/or quantitate receptor or binding sites in a cell or tissue. The compounds also can find use in vivo for studying the efficacy in the treatment of various diseases or conditions involving phosphatases, particularly YopH or PTP1B.

The present invention further provides a method of inhibiting PTB1B comprising contacting the PTP1B with an effective amount of a compound as described above. The present invention further provides a method of treating diabetes, e.g., type II diabetes, comprising administering an animal in need of treatment a compound as described above.

The potency of the compounds of the present invention to inhibit the YopH or PTP1B, can be determined by methods known in the art. See, for example, U.S. Pat. Nos. 5,688,992 and 6,307,090; WO 00/56760; and Chen et al. (supra).

It is contemplated that the compounds of the present invention also may be useful in preventing or prophylaxis of diseases mediated by SH2 domain binding, particularly diabetes, and infection by Yersinia.

The following examples further illustrate the invention but, of course, should not be construed as in any way limiting its scope.

### EXAMPLE 1

This Example illustrates a method of synthesis of embodiments of compounds of the present invention.

PTP-directed tripeptides were prepared using a standard Fmoc-based solid-phase strategy. The requisite N^{α}-Fmoc-protected pTyr mimetics were prepared. In the case of TB37, N-Fmoc-L-F₂Pmp-OH with free phosphonic acid was coupled using benzotriazole-1-yloxy-tris-(dimethylamino)-phosphonium hexafluoro-phosphate (BOP) ester formed by reacting N-Fmoc-L-F₂Pmp-OH, BOP, N,N-diisopropylamine, and HOBt in NMP. For TB32 and TB33, the 2-(trimethylsilyl)ethoxy) protecting group of the pTyr mimetic portion 14 was removed prior to peptide cleavage from the resin, by treatment with HF.pyr in THF:NMP (1:1) for 1 h. When tetrabutylammonium fluoride (TBAF, 1 M in THF) was used, N-terminal Fmoc group was also removed. As shown in FIG. 2, TB39 was obtained by attachment of Cbz-Glu(Ot-Bu)-OH in an analogous procedure using HOBt active ester from the intermediate 20. For the preparation of N-terminal phenethyl containing TB40, 20 was capped using a solution of 21 that obtained from the phenethyl alcohol by treatment of phosgene in THF (Org. Lett., 2(8), 1049-1051 (2000)), N-methylmorpholine (NMM) in N-methyl-2-pyrrolidinone (NMP). The final tripeptide-mimetics were purified to homogeneity by preparative RPHPLC. Analytical HPLC and proton NMR indicated a purity greater than 98%, and molecular weights were confirmed by FAB-MS.

General Synthetic Methods. Melting points were determined on a Mel Temp II melting apparatus and are uncorrected. Fast atom bombardment mass spectra (FABMS) were acquired with a VG Analytical 7070E mass spectrometer under the control of a VG 2035 data system. ¹H NMR data were obtained on a Varian 400 MHz or Bruker AC250 (250 MHz) instruments and are reported in ppm relative to TMS and referenced to the solvent in which they were run. Solvent was removed by rotary evaporation under reduced pressure and anhydrous solvents were obtained commercially and used without further drying. HPLCs were conducted using a Waters Prep LC4000 system having photodiode array detection and binary solvent systems as indicated where A=0.1% aqueous TFA and B=0.1% TFA in acetonitrile and either Vydac C18 (10μ) Peptide & Protein column (preparative size, 20 mm dia.×250 mm long with a flow rate of 10 mL/min; semipreparative size, 10 mm dia.×250 mm long, with a flow rate of 2 mL/min) or YMC J'sphere ODS-H80 (8 nM) column (preparative size, 20 mm dia.×250 mm long with a flow rate of 10 mL/min; analytical size, 4.6 mm dia.×250 mm long with a flow rate of 1 mL/min was used.

Peptide synthesis. PTP-directed tripeptides were prepared using Fmoc-based solid-phase synthesis. N^{α}-Fmoc derivatives of standard amino acids were obtained from Bachem Corp. (Torrence, Calif.) or Calbiochem-Novabiochem Corp. (San Diego, Calif.). The requisite phosphotyrosyl mimetics with orthogonal protection suitable for Fmoc-based chemistry were prepared. Each reaction was monitored by qualitative Kaiser test (E. Kaiser et al., Anal. Biochem., 34, 595 (1970)). In a representative procedure, a 0.1 milliequivalents N-Fmoc-Rink Amide resin (Bachem, 0.24 mmol/g) was swollen by with NMP, then the Fmoc protection was removed by treatment with 20% piperidine in NMP (1 mL for washing, then 1 mL; 20 min). The deblocked resin was washed well with NMP (10×2 mL) then coupled overnight with a solution of active ester formed by reacting 0.5 mmol each of N-Fmoc-L-Leu, 1-hydroxybenzotriazole (HOBt) and 1,3-diisopropylcarbodiimide (DIPCDI) in NMP (2 mL, 10 min. The resin was washed with NMP (5×2 mL) and dichloromethane (DCM, 5×2 mL), and the α-amino Fmoc-protection was removed by treatment with 20% piperidine in NMP (NMP (1 mL for washing, then 1 mL; 20 min). 0.25 mmole of N-Fmoc-pTyr mimetic was attached in a similar fashion using HOBt ester preactivation, followed by Fmoc removal. Subsequently, 0.5 mmol of N-Fmoc-Glu(Ot-Bu)-OH or N-Fmoc-Glu(OBn)-OH was attached in an analogous procedure using HOBt active ester. Peptides were cleaved from the resins by treatment of a mixture of TFA (1.85 mL), H₂O (0.1 mL) and triethylsilane (TES, 0.05 mL) for 1 h. The reaction mixture was filtered and the resin was rinsed with TFA (1 mL×3) and dichloromethane (1 mL×2). The combined filrate was concentrated and co-evaporated with H₂O to give crude solid which was purified by RPHPLC with a binary system of 0.1% TFA in H₂O and 0.1% TFA in acetonitrile.

Tripeptide mimetic TB21. (Purity>99% by RPHPLC; gradient elution from 20% to 90% of solvent B in 30 min, t_{R}=9.38 min). Mp 203° C. dec; ¹H NMR (DMSO-d₆) δ 8.01 (1H, d, J=7.6 Hz), 7.94 (1H, d, J=7.6 Hz), 7.84 (2H, d, J=7.6 Hz), 7.67 (2H, t, J=8.4 Hz), 7.47 (1H, d, J=7.6 Hz), 7.37 (2H, t, J=7.2 Hz), 7.28 (2H, t, J=7.2 Hz), 7.13 (1H, t, J=8.4 Hz), 7.02 (2H, m), 6.95 (1H, s), 6.85 (1H, s), 4.46 (1H, m), 4.18 (4H, m), 3.95 (1H, m), 2.97 (2H, m), 2.77 (1H, dd, J=9.2, 13.8 Hz), 1.76 (1H, m), 1.69 (1H, m), 1.52 (1H, m), 1.39 (2H, t, J=8 Hz), 0.80(3H, d, J=6.4 Hz) 0.75 (3H, d, J=6.8 Hz); FAB-MS (−VE) m/z 743 (M−H)⁻.

Tripeptide mimetic TB22. (Purity>99% by RPHPLC; gradient elution from 30% to 95% of solvent B in 30 min, t_{R}=7.40 min). Mp 196-198° C.; ¹H NMR (DMSO-d₆) δ 8.10 (1H, d, J=8.0 Hz), 8.02 (1H, d, J=8.0 Hz), 7.89 (2H, d, J=7.6 Hz), 7.83 (1H, s), 7.72 (2H, m), 7.50 (2H, d, J=8.4 Hz), 7.36 (5H, m), 7.14 (1H, d, J=7.6 Hz), 6.99 (1H, m), 6.88 (1H, s), 4.54 (1H, m), 4.20 (4H, m), 3.98 (1H, m), 3.85 (2H, d, J=4.4 Hz), 3.06 (1H, dd, J=4.8, 13.6 Hz), 2.82 (1H, m), 2.16 (1H, t, J=7.2 Hz), 1.79 (1H, m), 1.72 (1H, m), 1.54 (1H, m), 1.44 (2H, t, J=7.6 Hz), 0.85 (3H, d, J=6.8 Hz), 0.79 (3H, d, J=6.4 Hz); FAB-MS (−VE) m/z 729.5 (M−H)⁻.

Tripeptide mimetic TB23. (Purity>99% by RPHPLC; gradient elution from 20% to 90% of solvent B in 30 min, t_{R×}8.99 min). Mp 140-142° C.; ¹H NMR (DMSO-d₆) δ 8.04 (1H, d, J=7.6 Hz), 7.98 (1H, d, J=7.6 Hz), 7.89 (2H, d, J=7.6 Hz), 7.73 (1H, s, d, J=8 Hz), 7.55 (1H, d, J=8.0 Hz), 7.41 (2H, d, J=7.6 Hz), 7.32 (2H, m), 7.21 (4H, m), 7.01 (1H, s), 6.85 (1H, s), 4.57 (1H, s), 4.52 (1H, m), 4.24 (4H, m), 4.01 (1H, m), 3.00 (1H, m), 2.84 (1H, m), 2.21 (1H, t, J=7.6 Hz), 1.81 (1H, m), 1.73 (1H, m), 1.55 (1H, m), 1.43 (2H, t, J=7.2 Hz), 0.84 (3H, d, J=6.4 Hz), 0.80 (3H, d, J=6.4 Hz); FAB-MS (−VE) m/z 729 (M−H)⁻.

Tripeptide mimetic TB24. (Purity 98.3% by RPHPLC; gradient elution from 15% to 90% of solvent B in 30 min, t_{R}=13.46 min). Mp 187-189° C.; ¹H NMR (250 MHz, DMSO-d₆) δ 8.08 (2H, d, J=7.8 Hz), 7.94 (2H, d, J=7.3 Hz), 7.76 (2H, m), 7.60 (1H, d, J=7.3 Hz), 7.40 (8H, m), 7.02 (1H, s), 7.32 (2H, m), 4.61 (1H, m), 4.25 (4H, m), 4.02 (1H, m), 3.15 (1H, m), 2.97 (1H, m), 2.22 (2H, m), 1.87 (1H, m), 1.77 (1H, m), 1.53 (3H, m), 0.87 (3H, d, J=6.0 Hz), 0.82 (3H, d, J=5.5 Hz); FAB-MS (−VE) m/z 721.5 (M−H)

Tripeptide mimetic TB25. (Purity>99% by RPHPLC; gradient elution from 10% to 90% of solvent B in 30 min, t_{R}=9.73 min). Mp 197-199° C.; ¹H NMR (DMSO-d₆) δ 8.01 (2H, m), 8.99 (1H, d, J=8.0 Hz), 7.73 (2H, t, J=6.8 Hz), 7.55 (1H, d, J=7.6 Hz), 7.35 (8H, m), 7.15 (1H, s), 6.93 (1H, m), 4.54 (1H, dd, J=8.0, 13.8 Hz), 4.24 (4H, m), 4.00 (1H, m), 3.05 (1H, d, J-4, 14.2 Hz), 2.87 (1H, m), 2.21 (2H, m), 1.82 (1H, m), 1.71 (1H, m), 1.55 (1H, m), 1.44 (2H, t, J=7.2 Hz), 0.85 (3H, d, J=6.8 Hz), 0.80 (3H, d, J=6.4 Hz); FAB-MS (−VE) m/z 747 (M−H)⁻.

### EXAMPLE 2

This Example illustrates the properties of some of the compounds of the present invention.

TABLE 1
IC₅₀ Values in μM Against Various Enzymes

| Compound | PTB1B | YopH |
|---|---|---|
| TB 26 | 4.6 ± 2 | 2.8 ± 1.1 |
| TB 28 | 116 ± 20 | 82 ± 16 |
| TB 29 | 19.4 ± 3.6 | 19.4 ± 3.3 |
| TB 32 | 54.7 ± 15.3 | >100 |
| TB 33 | 8.3 ± 2.1 | 10.5 ± 3.9 |
| TB 34 | 3.1 ± 1.0 | 3.0 ± 0.7 |
| TB 35 | 44 ± 13 | 142 ± 51 |
| TB 36 | 1.5 ± 0.5 | 2.9 ± 0.4 |
| TB 37 | 1.41 ± 0.25 | 5.2 ± 2.2 |
| TB 39 | 72.0 ± 14 | 119 ± 14 |
| TB 40 | 58.2 ± 14.2 | 80.0 ± 8.5 |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A compound which is a tripeptide of the formula P-A-B-C or a prodrug thereof, wherein A is an amino acid having a carboxy C₁-C₆ alkyl group, B is tyrosine, phenylalanine, or a substituted tyrosine or phenylalanine, C is a hydrophobic amino acid, and P is an amine protecting group protecting the amine end of A; with the proviso that when A is Glu, P is fluorenylmethoxy carbonyl (Fmoc), B is p-phosphonomethyl, p-fluorophosphonomethyl, or p-difluorophosphonomethyl phenylalanine, or p-O-malonyl tyrosine, and C is Ala with a carboxylic acid or amide terminus, the carboxyalkyl group of A is not in acid form.

2. The compound of claim 1, wherein a carboxyl group is in the form of an ester, amide, carbonate, or urethane.

3. The compound of claim 1, wherein a carboxyl group is in the form of an ester.

4. The compound of claim 1, wherein the carboxy C₁-C₆ alkyl group of A is in the form of an ester.

5. The compound of claim 2, wherein the ester is a C₁-C₆ alkyl ester, aryl ester, aryl C₁-C₆ alkyl ester, C₁-C₆ alkyl aryl ester, hydroxy C₁-C₆ alkyl ester, halo C₁-C₆ alkyl ester, C₁-C₆ alkoxy C₁-C₆ alkyl ester, C₅-C₈ cycloalkyl ester, C₅-C₈ cyclic amine ester, C₁-C₆ alkanoyloxy C₁-C₆ alkyl ester, C₁-C₆ alkoxy carbonyloxy C₁-C₆ alkyl ester, cycloalkyl carbonyloxy C₁-C₆ alkyl ester, or 1,3-dioxolen-2-onyl C₁-C₆ alkyl ester, wherein the alkyl, cycloalkyl, and cyclic amino group may be optionally substituted by one or more of phenyl, heterocyclyl, C₁-C₆ alkyl, amino, C₁-C₆ alkylamino, C₁-C₆ dialkylamino, hydroxy, C₁-C₆ alkoxy, aryloxy, and benzyloxy.

6. The compound of claim 4, wherein the ester is a C₁-C₆ alkyl ester, aryl ester, aryl C₁-C₆ alkyl ester, C₁-C₆ alkyl aryl ester, hydroxy C₁-C₆ alkyl ester, halo C₁-C₆ alkyl ester, C₁-C₆ alkoxy C₁-C₆ alkyl ester, C₅-C₈ cycloalkyl ester, C₅-C₈ cyclic amine ester, C₁-C₆ alkanoyloxy C₁-C₆ alkyl ester, C₁-C₆ alkoxy carbonyloxy C₁-C₆ alkyl ester, cycloalkyl carbonyloxy C₁-C₆ alkyl ester, or 1,3-dioxolen-2-onyl C₁-C₆ alkyl ester, wherein the alkyl, cycloalkyl, and cyclic amino group may be optionally substituted by one or more of phenyl, heterocyclyl, C₁-C₆ alkyl, amino, C₁-C₆ alkylamino, C₁-C₆ dialkylamino, hydroxy, C₁-C₆ alkoxy, aryloxy, and benzyloxy.

7. The compound of claim 6, wherein the ester is an aryl C₁-C₆ alkyl ester.

8. The compound of claim 7, wherein the aryl C₁-C₆ alkyl ester is a benzyl ester.

9. The compound of claim 1, wherein A is Glu or Asp.

10. The compound of claim 8, wherein A is Glu or Asp.

11. The compound of claim 1, wherein B is a substituted tyrosine.

12. The compound of claim 1, wherein B is a substituted phenylalanine.

13. The compound of claim 11, wherein the substituted tyrosine is a tyrosine whose hydroxyl group and/or a ring hydrogen has been replaced or substituted with one, two, or more substituents selected from the group consisting of phosphono C₁-C₆ alkyl, phospho, phospho C₁-C₆ alkyl, phosphono halo C₁-C₆ alkyl, phosphono dihalo C₁-C₆ alkyl, carboxy C₁-C₆ alkyl, carboxy halo C₁-C₆ alkyl, carboxy C₁-C₆ alkoxy, carboxy, dicarboxy C₁-C₆ alkyl, dicarboxy halo C₁-C₆ alkyl, dicarboxy C₁-C₆ alkoxy, dicarboxy halo C₁-C₆ alkoxy, amino, amido, oxalylamino, C₁-C₆ alkylcarbonylamino, sulfo, sulfonyl, C₁-C₆ alkylthio, C₁-C₆ alkylsulfonyl, and halo C₁-C₆ alkyl sulfonyl.

14. The compound of claim 12, wherein the substituted phenylalanine is a phenylalanine having one, two, or more substituents selected from the group consisting of phosphono C₁-C₆ alkyl, phospho, phospho C₁-C₆ alkyl, phosphono halo C₁-C₆ alkyl, phosphono dihalo C₁-C₆ alkyl, carboxy C₁-C₆ alkyl, carboxy halo C₁-C₆ alkyl, carboxy C₁-C₆ alkoxy, carboxy, dicarboxy C₁-C₆ alkyl, dicarboxy halo C₁-C₆ alkyl, dicarboxy C₁-C₆ alkoxy, dicarboxy halo C₁-C₆ alkoxy, amino, amido, oxalylamino, C₁-C₆ alkylcarbonylamino, sulfo, sulfonyl, C₁-C₆ alkylthio, C₁-C₆ alkylsulfonyl, and halo C₁-C₆ alkyl sulfonyl.

15. The compound of claim 10, wherein B is a substituted tyrosine.

16. The compound of claim 10, wherein B is a substituted phenylalanine.

17. The compound of claim 15, wherein the substituted tyrosine is a tyrosine whose hydroxyl group and/or a ring hydrogen has been replaced or substituted with one, two, or more substituents selected from the group consisting of phosphono C₁-C₆ alkyl, phospho, phospho C₁-C₆ alkyl, phosphono halo C₁-C₆ alkyl, phosphono dihalo C₁-C₆ alkyl, carboxy C₁-C₆ alkyl, carboxy halo C₁-C₆ alkyl, carboxy C₁-C₆ alkoxy, carboxy, dicarboxy C₁-C₆ alkyl, dicarboxy halo C₁-C₆ alkyl, dicarboxy C₁-C₆ alkoxy, dicarboxy halo C₁-C₆ alkoxy, amino, amido, oxalylamino, C₁-C₆ alkylcarbonylamino, sulfo, sulfonyl, C₁-C₆ alkylthio, C₁-C₆ alkylsulfonyl, and halo C₁-C₆ alkyl sulfonyl.

18. The compound of claim 1, wherein C is a hydrophobic amino acid having a hydrophobic group selected from the group consisting of C₁-C₆ alkyl, C₁-C₆ haloalkyl, C₅-C₈ cycloalkyl, C₅-C₈ cycloalkyl C₁-C₆ alkyl, C₅-C₈ cycloalkoxy C₁-C₆ alkyl, aryl C₅-C₈ cycloalkyl, aryl, aryl C₁-C₆ alkyl, C₁-C₆ alkyl aryl, heterocyclyl, heterocyclyl C₁-C₆ alkyl, C₁-C₆ alkylthio C₁-C₆ alkyl, C₁-C₆ alkoxy C₁-C₆ alkyl, and hydroxyphenyl C₁-C₆ alkyl.

19. The compound of claim 10, wherein C is a hydrophobic amino acid having a hydrophobic group selected from the group consisting of C₁-C₆ alkyl, C₁-C₆ haloalkyl, C₅-C₈ cycloalkyl, C₅-C₈ cycloalkyl C₁-C₆ alkyl, C₅-C₈ cycloalkoxy C₁-C₆ alkyl, aryl C₅-C₈ cycloalkyl, aryl, aryl C₁-C₆ alkyl, C₁-C₆ alkyl aryl, heterocyclyl, heterocyclyl C₁-C₆ alkyl, C₁-C₆ alkylthio C₁-C₆ alkyl, C₁-C₆ alkoxy C₁-C₆ alkyl, and hydroxyphenyl C₁-C₆ alkyl.

20. The compound of claim 18, wherein C is selected from the group consisting of Ala, Val, Ile, Leu, Met, Phe, Tyr, Trp, and Nle.

21. The compound of claim 19, wherein C is a hydrophobic amino acid selected from the group consisting of Ala, Val, Ile, Leu, Met, Phe, Tyr, Trp, and Nle.

22. The compound of claim 1, wherein C is a hydrophobic amino acid having a C₁-C₆ alkyl group.

23. The compound of claim 22, wherein the carboxyl end of C is in the form of a carboxamide.

24. The compound of claim 23, wherein C is Ala or Leu in the form of a carboxamide.

25. The compound of claim 10, wherein C is a hydrophobic amino acid having a C₁-C₆ alkyl group.

26. The compound of claim 17, wherein C is a hydrophobic amino acid having a C₁-C₆ alkyl group.

27. The compound of claim 26, wherein the carboxyl end of C is in the form of a carboxamide.

28. The compound of claim 25, wherein C is Ala or Leu.

29. The compound of claim 26, wherein C is Ala or Leu.

30. The compound of claim 1, wherein P is an amine protecting group selected from the group consisting of an aryl C₁-C₆ alkoxy carbonyl, C₁-C₆ alkoxy carbonyl, carbobenzoxy, and carbamoyl.

31. The compound of claim 30, wherein P is an aryl C₁-C₆ alkoxy carbonyl.

32. The compound of claim 30, wherein the aryl C₁-C₆ alkoxy carbonyl is Fmoc.

33. The compound of claim 10, wherein P is an aryl C₁-C₆ alkoxy carbonyl.

34. The compound of claim 17, wherein P is an aryl C₁-C₆ alkoxy carbonyl.

35. The compound of claim 33, wherein the aryl C₁-C₆ alkoxy carbonyl is Fmoc.

36. The compound of claim 34, wherein the aryl C₁-C₆ alkoxy carbonyl is Fmoc.

37. The compound of claim 26, wherein P is an amine protecting group selected from the group consisting of an aryl C₁-C₆ alkoxy carbonyl, C₁-C₆ alkoxy carbonyl, carbobenzoxy, and carbamoyl.

38. The compound of claim 37, wherein P is an aryl C₁-C₆ alkoxy carbonyl.

39. The compound of claim 38, wherein the aryl C₁-C₆ alkoxy carbonyl is Fmoc.

40. The compound of claim 1, which has the formula I: 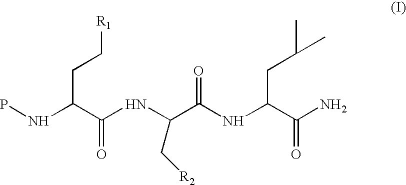
wherein P is selected from the group consisting of aryl C₁-C₆ alkoxy carbonyl, R₁ is aryl C₁-C₆ alkoxy carbonyl or aryloxy carbonyl, and R₂ is selected from the group consisting of carboxyaryl, carboxy C₁-C₆ alkoxy aryl, malonyloxyaryl, dicarboxy C₁-C₆ alkoxy aryl, carboxy-carboxy C₁-C₆ alkoxy aryl, halomalonyl aryl, carboxy-carboxy C₁-C₆ alkyl aryl, halomalonyloxy aryl, and dihalophosphono C₁-C₆ alkyl aryl.

41. The compound of claim 40, wherein P is selected from the group consisting of Fmoc, benzyloxy carbonyl, and phenylethyloxy carbonyl, R₁ is benzyloxy carbonyl or phenoxy carbonyl, and R₂ is selected from the group consisting of 4-carboxy phenyl, 4-carboxymethoxy phenyl, 4-malonyloxy phenyl, 3,4-dicarboxymethoxy phenyl, 3-carboxy-4-carboxymethoxy phenyl, 4-(α-fluoro malonyl) phenyl, 3-carboxy-4-carboxymethyl phenyl, 4-(α-fluoro malonyloxy) phenyl, and 4-(α,α-difluoro phosphonomethyl) phenyl.

42. The compound of claim 40, which has the formula Ia:

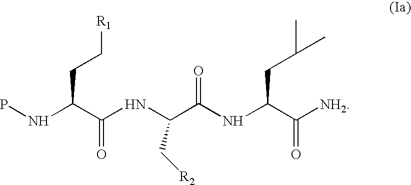

43. The compound of claim 42, wherein P is selected from the group consisting of Fmoc, benzyloxy carbonyl, and phenylethyloxy carbonyl, R₁ is benzyloxy carbonyl or phenoxy carbonyl, and R₂ is selected from the group consisting of 4-carboxy phenyl, 4-carboxymethoxy phenyl, 4-malonyloxy phenyl, 3,4-dicarboxymethoxy phenyl, 3-carboxy-4-carboxymethoxy phenyl, 4-(α-fluoro malonyl) phenyl, 3-carboxy-4-carboxymethyl phenyl, 4-(α-fluoro malonyloxy) phenyl, and 4-(α,α-difluoro phosphonomethyl) phenyl.

44. The compound of claim 43, wherein P is Fmoc, R₁ is benzyloxy carbonyl, R₂ is selected from the group consisting of 4-carboxymethoxy phenyl, 3-carboxy-4-carboxymethoxy phenyl, 4-(α-fluoro malonyl) phenyl, 3-carboxy-4-carboxymethyl phenyl, 4-(α-fluoro malonyloxy) phenyl, and 4-(α,α-difluoro phosphonomethyl) phenyl.

45. The compound of claim 43, wherein P and R₁ are benzyloxy carbonyl and R₂ is 4-carboxymethoxy phenyl.

46. The compound of claim 43, wherein P is phenylethyloxy carbonyl, R₁ is benzyloxy carbonyl, and R₂ is 4-carboxymethoxy phenyl.

47. A pharmaceutical composition comprising a compound of claim 1 and a pharmaceutically acceptable carrier.

48. A pharmaceutical composition comprising a compound of claim 40 and a pharmaceutically acceptable carrier.

49. A method of treating an animal exposed to Yersinia pestis comprising administering to the animal an effective amount of a compound which is a tripeptide of the formula P-A-B-C or a prodrug thereof, wherein A is an amino acid having a carboxy C₁-C₆ alkyl group, B is tyrosine, phenylalanine, or a substituted tyrosine or phenylalanine, C is a hydrophobic amino acid, and P is an amine protecting group protecting the amine end of A.

50. A method of inhibiting the protein-tyrosine phosphatase YopH of Yersinia pestis comprising contacting the Yersinia pestis with an effective amount of a compound which is a tripeptide of the formula P-A-B-C or a prodrug thereof, wherein A is an amino acid having a carboxy C₁-C₆ alkyl group, B is tyrosine, phenylalanine, or a substituted tyrosine or phenylalanine, C is a hydrophobic amino acid, and P is an amine protecting group protecting the amine end of A.

51. The method of claim 50, wherein the contacting is carried out in vivo.

52. The method of claim 50, wherein the contacting is carried out in vitro.

53. A method of treating diabetes in an animal comprising administering to the animal an effective amount of a compound which is a tripeptide of the formula P-A-B-C or a prodrug thereof, wherein A is an amino acid having a carboxy C₁-C₆ alkyl group, B is tyrosine, phenylalanine, or a substituted tyrosine or phenylalanine, C is a hydrophobic amino acid, and P is an amine protecting group protecting the amine end of A.
